(19) United States
(12) Patent Application Publication
(10) Pub. No.: US 2023/0273193 A1
(43) Pub. Date: Aug. 31, 2023

(54) SENSOR DEVICE, REAGENT FOR MODIFYING SURFACE OF SENSOR ELEMENT, METHOD OF MODIFYING SURFACE OF SENSOR ELEMENT AND METHOD OF MANUFACTURING SENSOR DEVICE

(71) Applicant: KABUSHIKI KAISHA TOSHIBA, Tokyo (JP)

(72) Inventor: Yoshiaki SUGIZAKI, Fujisawa Kanagawa (JP)

(73) Assignee: KABUSHIKI KAISHA TOSHIBA, Tokyo (JP)

(21) Appl. No.: 17/931,459

(22) Filed: Sep. 12, 2022

(30) Foreign Application Priority Data
Feb. 28, 2022 (JP) 2022-029831

(51) Int. Cl. G01N 33/543

(52) U.S. Cl. CPC G01N 33/54306

(57) ABSTRACT

According to one embodiment, a sensor device includes a sensor element formed of at least one selected from the group consisting of graphene, graphene oxide and carbon nanotubes and a modification molecule solid-phased on a surface of the sensor element via an anchor portion, and the anchor portion includes a first moiety containing a polycyclic aromatic ring or a polycyclic heteroaromatic ring and an electron-donating second moiety directly bonded directly to the first moiety.

### CROSS-REFERENCE TO RELATED APPLICATIONS

This application is based upon and claims the benefit of priority from Japanese Patent Application No. 2022-029831, filed Feb. 28, 2022, the entire contents of which are incorporated herein by reference.

### FIELD

Embodiments described herein relate generally to a sensor device, a reagent for modifying a surface of a sensor element, a method of modifying a surface of a sensor element and a method of manufacturing a sensor device.

### BACKGROUND

Sensor devices which can sense or capture various target substances or remove foreign substances are conventionally known.

In general, such sensor devices comprise a sensor element which supports a functional part such as a dye, a probe or the like on its surface. By bringing the functional part and a sample into contact with each other, the sensing or capture of the target substance is achieved.

Solid-phasing of the functional portion on the surface of the sensor element can be achieved by, for example, by bringing a reagent containing the functional portion into contact with the surface of the sensor element.

### BRIEF DESCRIPTION OF THE DRAWINGS

FIG. 1 is a diagram schematically showing an example of a sensor device according to the first embodiment.

FIG. 2 is a diagram schematically showing an example of a sensor device according to the second embodiment.

FIG. 3 is a diagram schematically showing an example of a sensor device according to the third embodiment.

FIG. 4 is a flowchart showing an example of a method of modifying a surface of a sensor element according to the fourth embodiment.

FIG. 5 is a flowchart showing an example of a method of manufacturing a sensor device according to the fifth embodiment.

FIG. 6 is a graph showing results of experiments carried out using Example 1 and comparative Example 4, together chemical formulas thereof.

FIG. 7 is a graph illustrating results of experiments carried out with use of Examples 1 to 4.

### DETAILED DESCRIPTION

In general, according to one embodiment, a technique for stably solid-phasing a modification molecule on a surface of a sensor element is provide.

Embodiments will be described hereinafter with reference to the accompanying drawings. Note that, throughout the embodiments, common structural elements are denoted by the same symbols and redundant explanations are omitted. Further, the drawings are schematic diagrams to facilitate understanding of the embodiments, and the shapes, dimensions, ratios, etc., may differ from actual conditions, but they may be redesigned as appropriate, taking into account the following descriptions and conventionally known technology.

(First Embodiment)

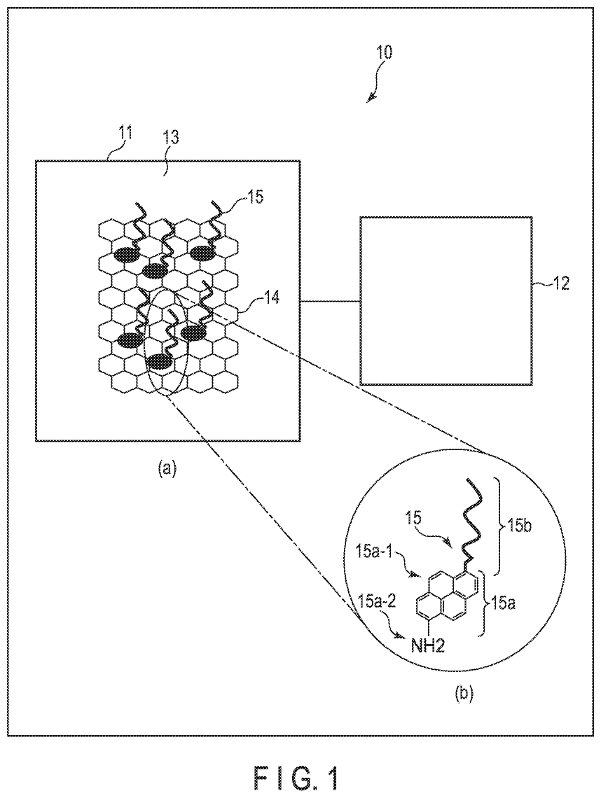

An example of a sensor device of the first embodiment will now be described with reference to FIG. 1. FIG. 1, part (a) is a diagram schematically showing a sensor device 10, and FIG. 1, part (b) is an enlarged view showing a structure of a modification molecule 15 solid-phased on a sensor element. As shown in FIG. 1, part (a), the sensor device 10 comprises a sensor element portion 11 and a housing portion 12 that is in liquid junction with the sensor element portion 11. The sensor element portion 11 has a base material 13, a sensor element 14 provided on a first surface of the base material 13 and a modification molecule 15 solid phased on a surface of the sensor element 14.

The sensor element 14 is formed from any one of graphene, graphene oxide and/or carbon nanotubes.

The base material 13 can be any material with any shape, which can support the sensor element 14, and preferably one which does not affect the reaction and the like in the sensor element. For example, it can be glass, plastic, quartz, silicon or the like, though it is not limited to these materials. The shape of the base material 13 can be a plate, sphere, rod, etc., or a plate or container with a recess, cup structure, groove structure and/or channel structure, etc., or a combination thereof.

As shown in FIG. 1, part (b), the modification molecule 15 includes an anchor portion 15a and an arbitrary functional portion 15b. The anchor portion 15a is adsorbed by interaction with the surface of the sensor element 14. Thereby, the solid phasing of the modification molecule 15 on the surface of the sensor element 14 is achieved. Note that FIG. 1, part (a) shows a situation where six modification molecules 15 are immobilized on the surface of the sensor element 14, but the arrangement and number of immobilized molecules are not limited to those of this case.

The anchor portion 15a comprises a first moiety and a second moiety. The first moiety is a portion where there are delocalized n-electrons. The second moiety is a site including the first moiety and an electron-donating portion directly coupled to the first moiety.

The first moiety is a polycyclic aromatic ring and a polycyclic heteroaromatic ring and the like. The first moiety is adsorbed on the surface of the sensor element 14 by ππ interactions. Note that an aromatic ring is a conjugated unsaturated ring structure with 4n+2 (n is a natural number greater than or equal to 1) π-electrons.

Examples of the polycyclic aromatic ring include anthracene, tetracene, pentacene, benzopyrene, chrysene, pyrene, triphenylene, corannulene, coronene and ovarene, as listed below, but the examples are not limited to these.

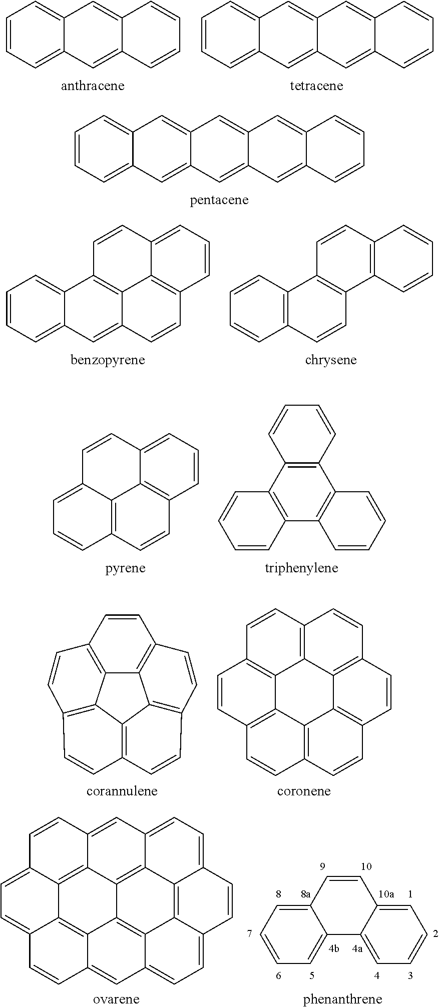

Examples of the polycyclic heteroaromatic ring include indole, isoindole, benzoimidazole, purine, benzotriazole, quinoline, isoquinoline, quinazoline, quinoxaline, cynnoline, pteridine, chromene (benzopyran), isochromene (benzopyran), acridine, xanthene, carbazole and benzo-C-synnoline(en) as listed below, but not limited to these.

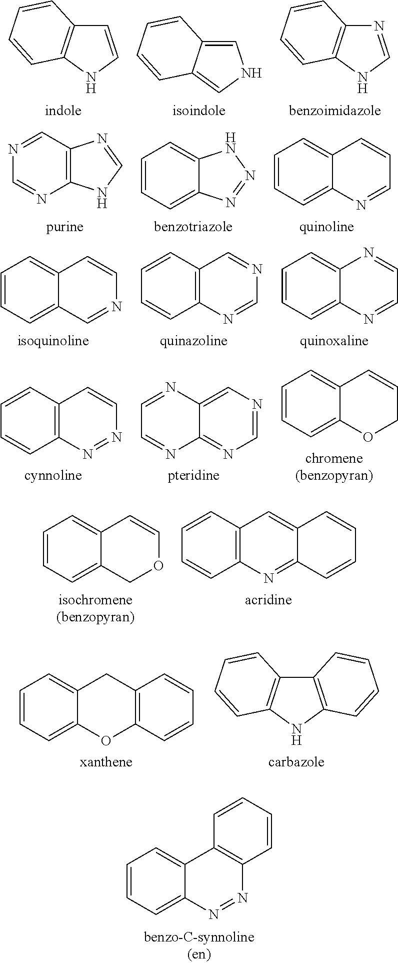

The second moiety is a electron-donating moiety or functional group bonded directly to the aromatic carbons. The electron-donating moiety is, for example, an alkyl group, a phenyl group or a derivative thereof, which exhibits electron-donating properties by an inductive effect. The second moiety should more preferably be a structure or functional group which exhibits electron-donating properties by a resonance effect.

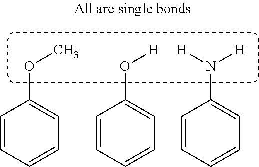

Specifically, the second moiety is, for example as follows.

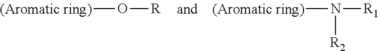

where R, R₁ and R₂ are hydrogen, hydrocarbons and derivatives thereof, etc. More specifically, the second moiety has a structure in which an oxygen or nitrogen atom thereof is bonded directly to the aromatic carbon and the oxygen or nitrogen atom is single-bonded to each one of R, R₁ and R₂. The second moiety has a structure in which the oxygen or nitrogen atom has no double or triple bond with any one of R, R₁ and R₂. The second moiety is, for example, a hydroxy group, an amino group, an alkoxy group, an alkylamino group or a dialkylamino group. The second moiety may be of a structure in which the oxygen or nitrogen shown in the chemical formula 4 is conjugated with the aromatic ring to form a double bond with a carbon atom which constitutes the aromatic ring. The second moiety which forms a double bond may be of a structure in which any one of R, R₁ and R₂ is detached.

Due to the presence of the anchor portion 15a with such a structure as described above, a strong ππ interaction can be exerted with the sensor element portion. The ππ interaction is enhanced by the presence of an electron-donating substituent, and by the resonance effect, which is created by direct bonding to the aromatic carbon, the effect of the interaction is even further enhanced.

As described above, the modification molecule 15 includes, in addition to the anchor portion 15a, an arbitrary functional portion 15b. The functional portion 15b may be selected according to the usage of the sensor device. For example, the functional portion may be a portion which captures a specific substance, or may be a portion which exhibits a catalytic action for a specific chemical reaction, or may be a portion to which a specific substance does not easily adhere. In other words, the functional portion 15 is a portion having the function of capturing a specific substance, a portion of the function of exhibiting a catalytic action for a specific chemical reaction, or a portion having the function of being hard for a specific substance to adhere.

Between the anchor portion 15a and the functional portion 15b, a spacer portion may further be present. The bonding between the anchor portion 15a and the functional portion 15b is chemically achieved in a position where the properties of the anchor portion 15a described above are not interfered with, that is, a position where the π-electron density and resonance effect are not affected, according to the chemical structure and by a conventional method known per se. Further, electron-attractive atomic groups may as well be included therein as long as they do not cancel out the electron-donating properties. The spacer portion is, for example, an amino group, an alkoxy group, peptide, polyethylene glycol, hydrocarbon group or a derivative thereof.

In the case where the function is to capture a specific substance, examples of the functional portion 15b are nucleic acids such as DNA aptamer and RNA aptamer, a peptide, an antibody, lectin, avidin, biotin, sialic acid and sugar chain, which can be one of any binding pair which has specific affinity with respect to each other.

In the case where the function is a catalytic action for a particular chemical reaction, examples of functional portions 15b can be various enzymes and the like. Further, they may as well be ribozyme and deoxyribozyme, which are nucleic acids having enzymatic activity, but the examples are not limited to these.

In the case where the function is to make it difficult for a specific substance to adhere, it suffices if the functional portion 15b has a structure which makes it difficult for the specific substance to adhere to a particular site. Examples thereof can be hydrophilic substances such as polyethylene glycol and the like and bipolar substances such as phospholipids and sulfobetaine. The structure which makes it difficult for a particular substance to adhere to its site may as well be referred to as a blocking agent.

The housing portion 12 may be a space which provides a reaction portion for the sensor element portion 11. Further, the housing portion 12 may comprise a flow path used to deliver liquids such as test objects, reagents and/or cleaning liquid or gases, to the sensor element portion 11 or to collect liquids from the sensor element portion 11, housing means to contain those liquids and gases, a transport mechanism to move those liquids by pushing or suctioning the liquid out, and/or a control mechanism to control the movement of the liquids, and the like. The substances to be contained in the housing portion 12 may be liquids, gases, or mixtures thereof.

According to the first embodiment described above, a sensor device can be provided in which modification molecules can be firmly solid-phased in the sensor element portion. Such a sensor device can as well exhibit an effect that it is robust against foreign substances.

(Second Embodiment)

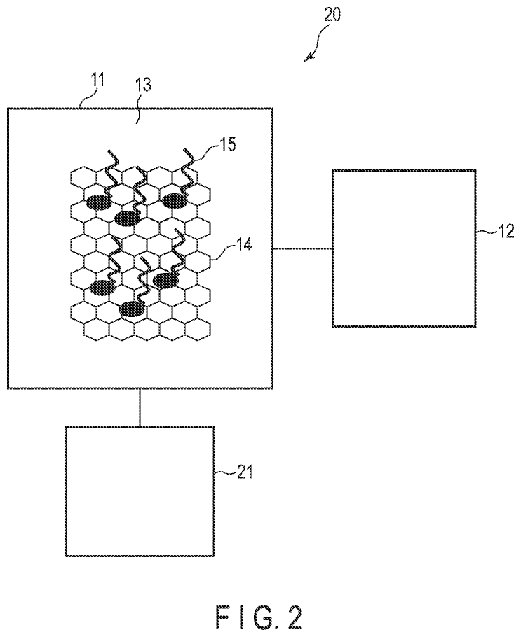

FIG. 2 shows an example of a sensor device of the second embodiment. A sensor device 20 may have a structure similar to that of the sensor device of the first embodiment, except that it further comprises a signal collection portion 21. The signal collection portion 21 can be a communication line to collect signals generated from the sensor element section 11, that is, an optical path, a conductive material or the like. Further, the signal collection portion 21 may further comprise a connection mechanism to be connected to a detector which reads signals from the sensor device.

According to the second embodiment described above, a sensor device can be provided in which a modification molecule is firmly solid-phased on the sensor element portion. Such a sensor device may be well exhibit a robust effect.

(Third Embodiment)

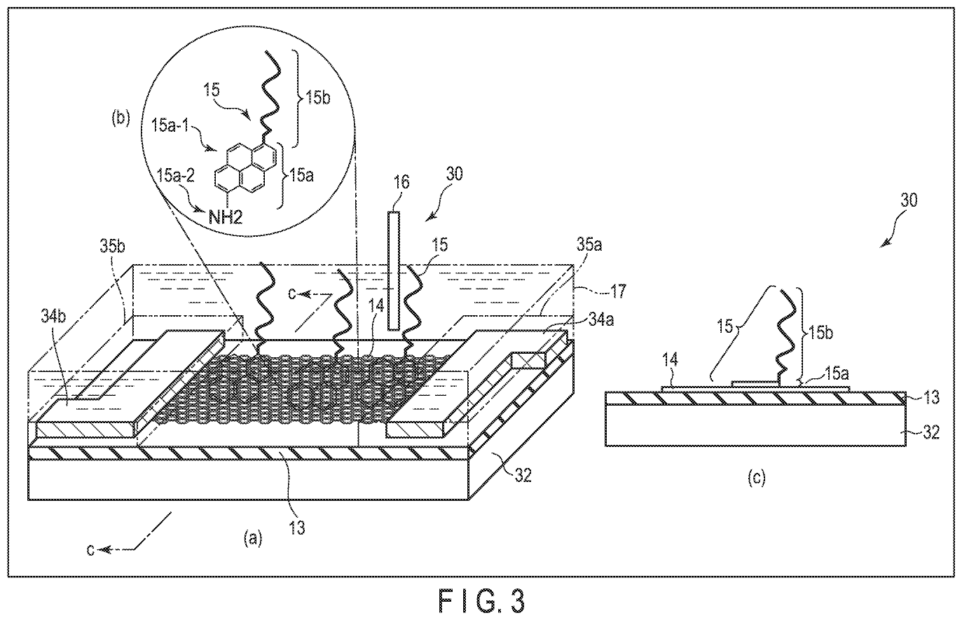

FIG. 3 schematically shows an example of a sensor device according to the third embodiment. FIG. 3, part (a) is a perspective view of a sensor device 30, FIG. 3, part (b) is an enlarged view of a modification molecule 15 solid-phased on a sensor element, and FIG. 3, part (c) is a cross-sectional view of the sensor device 30 taken along line c-c in FIG. 3, part (a).

The sensor device 30 comprises a sensor element 14 on a base material 13 formed on a substrate 32. A solid-phased modification molecule 15 is supported on the surface of the sensor element 14. The sensor element 14 functions as a channel. At both ends of the sensor element 14, conductors for acquiring signals from the sensor element 14, that is, for example, metal plates 34a and 34b are disposed so as to be in contact with the sensor element 14. The metal plates 34a and 34b function as source or drain electrodes. The metal plates 34a and 34b are covered by coating members 35a and 35b formed of an insulating material. The sensor device 30 includes a gate electrode 16 which applies a potential to the sensor element 14. The gate electrode 16 can be provided as a back gate connected to the substrate 32. In the case where the sensor device 30 is a device for sensing a liquid 17 in contact with the sensor element 14 as an object, the gate electrode 16 can be provided above the sensor element 14 so that a voltage is applied to the sensor element 14 via the liquid 17. The sensor device 30 can detect an interaction which occurs between the modification molecule 15 and the object to be inspected, on the sensor element 14, for example, from the change in drain electrical characteristics obtained when a voltage is applied between the metal plates 34a and 34b. For example, when the modification molecule 15 binds to a specific substance having polarity, contained in the object to be inspected, a minute electric field is applied to the sensor element and the drain current characteristics change.

According to the third embodiment described above, a sensor device can be provided in which a modification molecule is firmly solid-phased on the sensor element portion. Such a sensor device can exhibit a robust effect as well.

(Fourth Embodiment)

As the fourth embodiment, a sensor element surface modification reagent is provided, which solid-phase a modification molecule on a sensor element surface of a sensor device. The sensor element to be modified by the sensor element surface modification reagent is a sensor element comprising at least one selected from the group consisting of graphene, graphene oxide and carbon nanotubes.

The sensor element surface modification reagent includes a modification molecule 15 having an anchor portion 15a as described above. That is, the sensor element surface modification reagent contains a modification molecule including an anchor portion 15a, which further includes a first moiety 15a-1 where there are delocalized π-electrons and an electron-donating second moiety directly bonded to the first moiety. Via the anchor portion 15a, the modification molecule is solid-phased on the surface of the sensor element 14.

According to the sensor element surface modification reagent of the embodiment, a ππ interaction stronger than that of the conventional technique can be obtained due to the configuration of the anchor portion 15a. Therefore, even if the surface of the sensor element 14 to be modified is contaminated by foreign substances, the modification molecule 15 can be firmly solid-phased on the surface of the sensor element 14. In addition, in order to improve the solid phase density, it is conventionally necessary to increase, for example, the concentration of the reagent such as a probe solution. However, in this example, it is possible to solid-phase the modification molecules at high density without increasing the concentration of the reagent.

As described above, the modification molecule can further include an arbitrary functional portion 15b in addition to the anchor portion 15a.

The sensor element surface modification reagent may be provided in a state that the modification molecules 14 are contained in a solution, or as modification molecules in a dry state. They may be provided in appropriate containers, respectively. Further, the sensor element surface modification reagent may as well contain a component for the stability of the functional portion, that is, for example, a stabilizer such as a salt, peptide or the like. For example, the solution for the sensor element surface modification reagent may be an aqueous solution, an organic solution or a mixture thereof, depending on the modification molecule 14.

According to the fourth embodiment described above, a sensor element surface modification reagent is provided, which can stably solid-phase the modification molecule 15 to the sensor element 14. With use of such a sensor element surface modification reagent, the sensor device thus provided can also exhibit a robust effect against foreign substances.

(Fifth Embodiment)

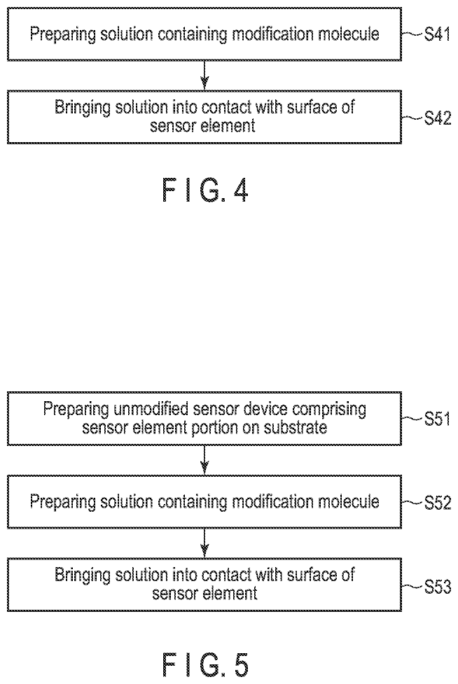

As the fifth embodiment, a method of modifying a surface of the sensor element of the previous embodiment is provided. As shown in FIG. 4, the method of modifying the surface of the sensor element comprises: preparing a solution containing a modification molecule (S41); and dropping the solution onto the surface of the sensor element (S42). The modification molecule contains an anchor portion. The anchor portion includes a first moiety where there are delocalized π-electrons and an electron-donating second moiety directly bonded to the first moiety. The details thereof are as described above.

After dropping the solution, the surface of the sensor element is left at room temperature, and may be then washed and dried as necessary.

According to the method of modifying the surface of the sensor element according to the embodiment, a stronger ππ interaction than that of the conventional techniques can be obtained by the configuration of the anchor portion. Therefore, even if there is contamination on the surface of the sensor element to be modified, the modification molecule can be firmly solid-phased on the sensor element surface. For example, even when there are residuals created from the polyimide protective film formed on the surface of the sensor element, the solid phase can be stably achieved. Further, with the conventional techniques, for example, the concentration of a reagent such as the probe solution need to be increased to improve the solid phase density. However, it is now possible with the present embodiment to solid-phase modification molecules at high density without increasing the concentration of the reagent.

As described above, according to the fifth embodiment, a method for modifying the surface of a sensor element, which can stably solid-phase a modification molecule onto the sensor element is provided. With such a method of modifying the surface of a sensor element, the sensor device thus provided can also exhibit a robust effect against foreign substances.

(Sixth Embodiment)

As the sixth embodiment, a method of manufacturing a sensor device is provided. As shown in FIG. 5, the method of manufacturing a sensor device comprises the following steps: preparing an unmodified sensor device comprising a sensor element portion on a substrate (S51); preparing a solution containing a modification molecule (S51); and dropping the solution onto the surface of the sensor element (S53).

It suffices if the unmodified sensor device is of a state in which the modification molecule is not solid-phased on the sensor element portion, and may be prepared by any of the means and processes known per se, as desired.

With the manufacturing method of the sixth embodiment, a sensor device which is, for example, any one of the first to third embodiments as described above is provided. That is, a sensor device in which a modification molecule is firmly solid-phased on the sensor element portion is provided. The sensor device provided by such a manufacturing method can also exhibit a robust effect against foreign substances.

The method includes solid-phasing a modification molecule on a surface of a sensor element. The sensor element is formed from at least one selected from the group consisting of graphene, graphene oxide and carbon nanotubes. The modification molecule includes an anchor portion. The anchor portion includes a first moiety where there are delocalized n-electrons and an electron-donating second moiety directly bonded to the first moiety. The details thereof are as described above. The solid phasing includes preparing a solution containing the modification molecule, and bringing (for example dropping) the solution into contact with the surface of the sensor element.

(Example 1)

Measurement of FET response of graphene solid-phased with xanthene ring, which is a fluorescent dye

A Rhodamine 6G (R6G) aqueous solution and an Alexa 488 aqueous solutions were prepared respectively. The aqueous solutions each contain 1 mM-HEPES and 1 mM-KCL.

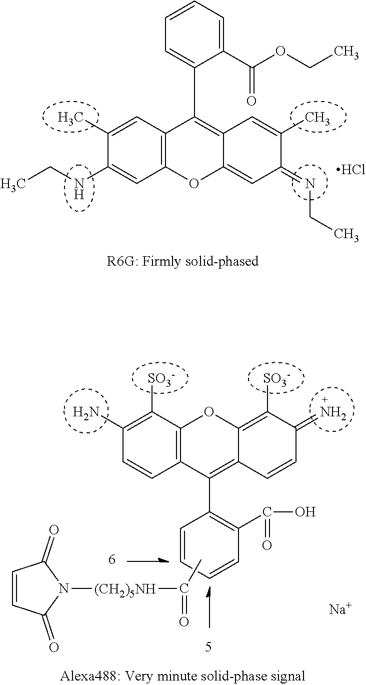

On the surface of each of sensor elements formed of graphene, a polyimide protective film was formed, and the graphene-exposed surface was treated with 1 mM-HEPES and 1 mM-KCL. Onto the graphene-exposed surfaces, the previously prepared aqueous solutions of Rhodamine 6G (R6G) and Alexa 488 were dropped, respectively. Thereafter, the surfaces were left at room temperature for 15 minutes to allow Rhodamine 6G and Alexa 488 to be solid-phased on the surfaces of the sensor elements as modification molecules, respectively.

Then, after washing each sensor element, the rate of change in drain current was measured for Rhodamine 6G (Example 1) and Alexa 488 (Comparative Example 1). The drain current change rate was obtained by measuring the current value of graphene at different gate voltages. The drain current of graphene shows a V-shaped characteristics when plotted against the gate voltage, and the bottom of the V-shape is called the charge neutral point. Here, it is known that positive holes conduct as carriers at voltages lower than the charge neutral point. In this experiment, the gate voltage at which the gate voltage dependency is at the highest in the hole conduction region was used for evaluation. More specifically, the measurements were carried out at a gate voltage 100 mV lower than the charge neutral point to obtain results, and the results indicated that it was 400 mV for Rhodamine 6G, and 500 mV for Alexa 488. Note that the drain current changes as the modification molecule is solid phased.

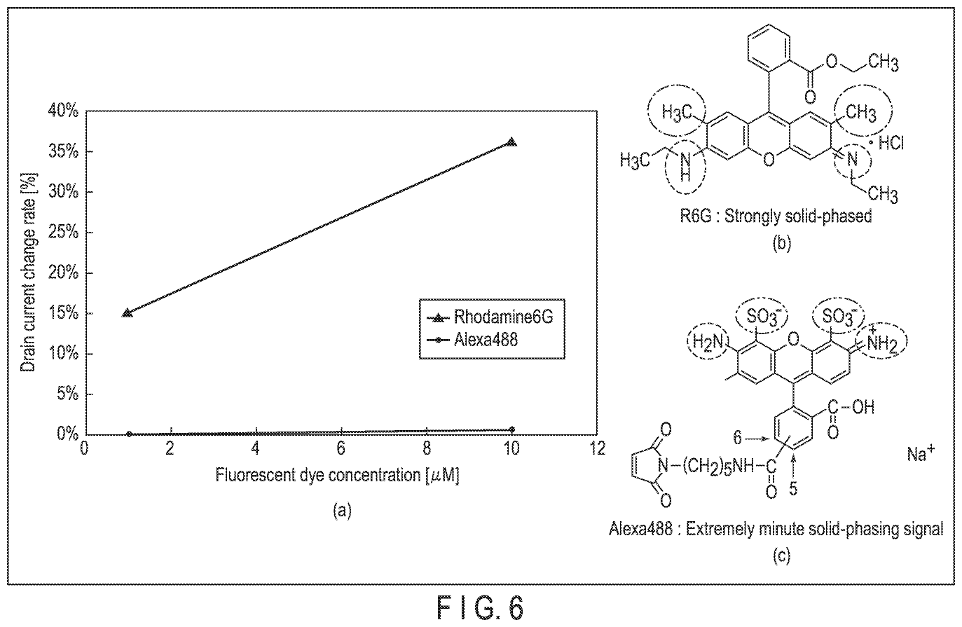

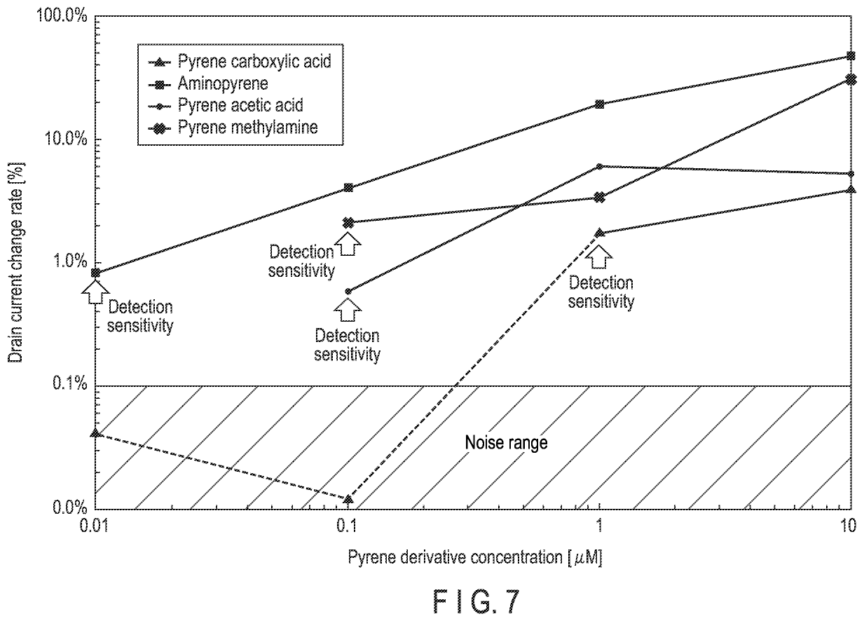

The results are shown in the graph in FIG. 6. The drain current change rate by Rhodamine 6G (Example 1) is indicated by a triangle, and a large increase depending on an increase in concentration of Rhodamine 6G was observed. On the other hand, in the case of Alexa 488 indicated by a circle in the graph, no substantial change in drain current was detected, and further, no substantial change in drain current along with concentration was observed.

FIG. 6, part (b) shows the chemical structure of Rhodamine 6G (R6G), and FIG. 6, part (c) shows the chemical structure of Alexa 488. As shown, in Rhodamine 6G (R6G), two electron-donating groups are present in the sites encircled by dotted lines (----). By contrast, in Alexa 488, there are electron-donating groups in the sites encircled by dotted lines (----) and SO3—as electron-withdrawing groups. The SO3-groups are encircled by other dotted lines (--..--), respectively.

In other words, in Rhodamine 6G, two amines, each exhibiting the electron-donating properties due to the resonance effect, are directly bonded to the xanthene ring. The amines are surrounded by the dotted lines (----). Further, two methyl groups are bonded thereto exhibit the electron-donating properties due to the induction effect, which are encircled by the dotted lines (-.-.-) in the figure. Due to this structure, Rhodamine 6G has an even higher π-electron density in the xanthene ring.

On the other hand, in Alexa 488, two amines, each exhibiting electron-donating properties due to the resonance effect, and two sulfonic acid groups, each exhibiting electron-withdrawing properties similarly due to the resonance effect, are bonded to the xanthene ring. Therefore, in Alexa 488, the electron pairs donated from the electron-donating group (amine) to the xanthene ring (aromatic ring) due to the resonance effect, are unevenly distributed to the electron-withdrawing groups (sulfonic acid groups), and the π-electron density of the xanthene ring is canceled out. As a result, it was considered that the ππ interaction between the xanthene ring and graphene was stronger in Rhodamine 6G. From these results, it has been found that when there is contamination on the surface of the graphene sensor element, sufficient solid phasing cannot be obtained in the comparative example, whereas strong solid phasing can be achieved in this example. This result was also confirmed as to the robustness demonstrated by the embodiments. Even when functional groups exhibiting electron-withdrawing properties by the resonance effect (that is, for example, carbonyl group, cyano group, nitro group or sulfonyl group) are bound to the aromatic ring, the number of functional groups (or secondary moieties) exhibiting electron-donating properties by the resonance effect and bonded to the aromatic ring is larger than the number of functional groups exhibiting electron-withdrawing properties by the resonance effect and bonded to the aromatic ring. It is thus considered that the electron-withdrawing effect by the resonance effect is canceled out, thereby increasing the electron density of the aromatic ring.

(Example 2, Comparative Examples 2 to 4)

Measurement of FET response of graphene on which various pyrene derivatives were solid-phased as modification molecules

Based on the results of the previous experiment, it was considered that Rhodamine 6G involves a cation-π interaction with graphene because Rhodamine 6G is a cation and Alexa 488 is an anion. Therefore, to investigate the influence of the π-electron density and the cation-π interaction, a further experiment was carried out with regard to a case where an amino group and a carboxylic acid group were bonded to pyrenes, which is polycyclic aromatic groups, to form cations and anions, respectively. Further, the difference in the response of graphene FETs was examined for the case where the π-electron density was greatly changed by the resonance effect in which an amino group and a carboxylic acid group were bonded to pyrenes directly, and for the case where the change in π-electron density was suppressed by blocking the resonance effect, in which one carbon atom is interposed in the bond. More specifically, the following experiments were conducted.

Aqueous solutions of pyrene carboxylic acid (Comparative Example 2), aminopyrene (Example 2), pyrene acetic acid (Comparative Example 3) and pyrene methylamine (Comparative Example 4) were prepared and they were solid-phased as modification molecules 15 on the surfaces of the sensor elements 14 formed of graphene, respectively, by a method similar to that of Example 1. The chemical formulas and some characteristics of these compounds are shown in Table 1.

TABLE 1

| | |
|---|---|
| 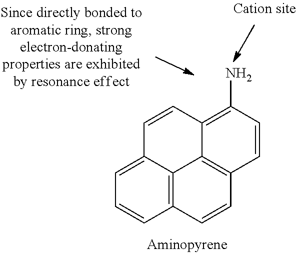 [SMILES: Nc1ccc2ccc3cccc4ccc1c2c34] | 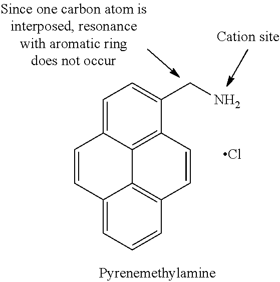 [SMILES: NCc1ccc2ccc3cccc4ccc1c2c34 ; Cl] |
| 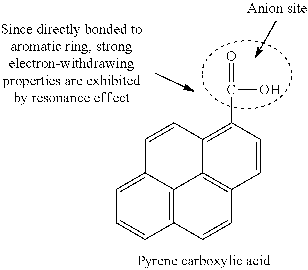 [SMILES: O=C(O)c1ccc2ccc3cccc4ccc1c2c34] | 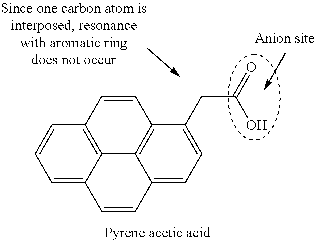 [SMILES: O=C(O)Cc1ccc2ccc3cccc4ccc1c2c34] |

The drain currents were measured for Example 2, Comparative Example 2, Comparative Example 3 and Comparative Example 4, respectively.

The results are shown in FIG. 5. FIG. 5 shows a double logarithmic graph in which the horizontal axis indicates the concentration of each pyrene derivative (μM) and the vertical axis indicates the drain current change rate (%). In the graph, results with pyrene carboxylic acid (Comparative Example 2) are shown by triangles, those from aminopyrene (Example 2) by squares, those from pyrene acetic acid (Comparative Example 3) as circles, those from pyrene methylamine (Comparative Example 4) by crosses.

A range of 0.1% or less in drain current change rate is a range of noise, in which in which levels corresponding to detection sensitivity cannot be obtained. The detection sensitivity indicates a condition of the lowest pyrene derivative concentration among conditions of pyrene derivatives with which a valid drain current change rate was detected. Aminopyrene (Example 2: squares) exhibited the best result out of the four derivatives, and good detection sensitivities were obtained from concentrations as low as 0.01 μM. Then, the drain current change rate increased substantially linear in the double logarithmic graph in a concentration-dependent manner as the concentration was increased as 0.1 μM, 1 μM, to 10 μM. From these results, it is clear that aminopyrene can stably achieve solid-phase from low concentrations. For pyrene acetic acid (Comparative Example 3: circles) and pyrene methylamine (Comparative Example 4: crosses), similar drain current change rates were observed. For pyrene acetic acid (Comparative Example 3: circles), it peaked at 1 μM, and the drain current change rate decreased at 10 μM. For pyrene methylamine (Comparative Example 4: crosses), it reached a detection sensitivity at 0.1 μM, the drain current change rate remained substantially unchanged up to 1 μM, and the drain current change rate increased slightly at 10 μM. For pyrenecarboxylic acid (Comparative Example 2), the drain current change rate remained within the noise range from the concentration from 0.01 μM to 0.1 μM. Then, it reached a detection sensitivity at 1 μM; however, the drain current change rate remained low at 1.0% even in a range of 10 μM.

Aminopyrene, with which the π-electron density of pyrene was increased by directly bonding an amine, exhibited the strongest response of graphene FETs. Pyrenecarboxylic acid which the π-electron density of pyrene was decreased by directly bonding carboxlic acid, exhibited the weakest response of graphene FETs. Both pyrene methylamine and pyrene acetic acid, with which the resonance effect was suppressed by interposing one carbon, were not as strong as the response of aminopyrene, and a difference of 10 times or more was observed with pyrene acetic acid and further, a difference of about 2 times was observed with pyrene methylamine.

From these results, it has been confirmed that atomic groups with increased π-electron density due to the resonance effect exhibit the ability to bond very strongly to graphene.

As described above, according to the embodiment, it has been demonstrated that a sensor device in which the modification molecule is firmly solid-phased on the sensor element portion can be provided.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A sensor device comprising:
a sensor element formed of at least one selected from the group consisting of graphene, graphene oxide and carbon nanotubes; and
a modification molecule solid-phased on a surface of the sensor element via an anchor portion,
the anchor portion including a first moiety containing a polycyclic aromatic ring or a polycyclic heteroaromatic ring and an electron-donating second moiety directly bonded directly to the first moiety.

2. The sensor device of claim 1, wherein
the second moiety contains at least one selected from the group consisting of an amino group, a hydroxy group, an alkoxy group, an alkylamino group and a dialkylamino group.

3. The sensor device of claim 1, wherein,
the first moiety is selected from the group consisting of anthracene, tetracene, pentacene, benzopyrene, chrysene, pyrene, triphenylene, corannulene, coronene, ovalene, indole, isoindole, benzoimidazole, purine, benzotriazole, quinoline, isoquinoline, quinazoline, quinoxaline, cynoline, pteridine, chromene, isochromene, acridine, xanthene, carbazole and benzo-C-cynoline(en).

4. The sensor device of claim 2, wherein,
the first moiety is selected from the group consisting of anthracene, tetracene, pentacene, benzopyrene, chrysene, pyrene, triphenylene, corannulene, coronene, ovalene, indole, isoindole, benzoimidazole, purine, benzotriazole, quinoline, isoquinoline, quinazoline, quinoxaline, cynoline, pteridine, chromene, isochromene, acridine, xanthene, carbazole and benzo-C-cynoline(en).

5. The sensor device of claim 1, wherein
the modification molecule further comprises a functional portion bonded to the anchor portion.

6. The sensor device of claim 2, wherein
the modification molecule further comprises a functional portion bonded to the anchor portion.

7. The sensor device of claim 3, wherein
the modification molecule further comprises a functional portion bonded to the anchor portion.

8. The sensor device of claim 5, wherein
the functional portion has a function of capturing a specific substance, a function of specifically bonding to a specific substance, or a function of making it difficult for the specific substance to adhere.

9. The sensor device of claim 6, wherein
the functional portion has a function of capturing a specific substance, a function of specifically bonding to a specific substance, or a function of making it difficult for the specific substance to adhere.

10. The sensor device of claim 7, wherein
the functional portion has a function of capturing a specific substance, a function of specifically bonding to a specific substance, or a function of making it difficult for the specific substance to adhere.

11. A reagent for modification of a surface of a sensor element,
containing a modification molecule including an anchor portion including a first moiety, which is a polycyclic aromatic ring or a polycyclic heteroaromatic ring and an electron-donating second moiety directly bonded to the first moiety,
the reagent for modification of a surface of a sensor element solid-phasing the modification molecule via the anchor portion onto the surface of the sensor element formed of at least one selected from the group consisting of graphene, graphene oxide and carbon nanotubes.

12. The modification reagent of claim 11, wherein the modification molecule further comprises a functional portion bound to the anchor portion.

13. A method of modifying a surface of a sensor element, comprising:
preparing a solution containing a modification molecule;
bringing the solution into contact with the surface of the sensor element, the sensor element being formed of at least one selected from the group consisting of graphene, graphene oxide, and carbon nanotubes; and
modifying the surface of the sensor element with the modification molecule, the surface of the sensor element including an anchor portion including a first moiety, which is a polycyclic aromatic ring or a polycyclic heteroaromatic ring and an electron-donating second moiety directly bonded to the first moiety.

14. A method of manufacturing a sensor device comprising a sensor element and a modification molecule solid-phased onto a surface of the sensor element, the sensor element being formed from at least one selected from the group consisting of graphene, graphene oxide, and carbon nanotubes, and the modification molecule including an anchor portion including a first moiety, which is a polycyclic aromatic ring or a polycyclic heteroaromatic ring and an electron-donating second moiety directly bonded to the first moiety,
the method comprising:
preparing a solution containing the modification molecule; and
bringing the solution into contact with the surface of the sensor element.
